(19) United States
(12) Patent Application Publication
(10) Pub. No.: US 2009/0137969 A1
(43) Pub. Date: May 28, 2009

(54) Apparatus and method for sterilizing a tubular medical line port

(76) Inventors: Anthony J. Colantonio, Meadville, PA (US); Menno D. Jager, Meadville, PA (US)

Correspondence Address: Floyd B. Carothers;CAROTHERS AND CAROTHERS, Suite 200, 445 Fort Pitt Blvd., Pittsburgh, PA, 15219, US

(21) Appl. No.: 12/077,881

(22) Filed: Mar. 20, 2008

Related U.S. Application Data
(60) Provisional application No. 60/996,558, filed on Nov. 26, 2007.
(60) Provisional application No. 61/006,889, filed on Feb. 5, 2008.

(51) Int. Cl. A61M 25/00; A61L 2/16

(52) U.S. Cl. 604/265; 422/28

(57) ABSTRACT

A device for sterilizing a tubular medical line IV port. The device includes a cap having an open tubular cavity on a distal end which is dimensioned to receive the medical line port therein. A handle is provided on the proximal end of the device for grasping and manipulating the cap. An outer disinfectant-impregnated member is coaxially secured in the distal cavity and dimensioned for engaging and scrubbing exterior surfaces or threads of the tubular port when inserted into the cavity. An inner disinfectant-impregnated member may also be coaxially secured in the cavity to simultaneously scrub internal surfaces of the port.

### CROSS REFERENCE

This application is based on U.S. Provisional Patent Application No. 60/996,558, filed Nov. 26, 2007, and U.S. Provisional Patent Application 61/006,889, filed Feb. 5, 2008.

### FIELD OF THE INVENTION

The present invention relates to the field of medical liquid administration, and more particularly, to an apparatus and method for applying antiseptic to the female end of a medical infusion apparatus.

### BACKGROUND OF THE INVENTION

Improvements in patient safety have been among the primary concerns of many efforts in today's healthcare industry. Healthcare associated infections remain a major area of focus for these efforts. The Center for Disease Control and Prevention cites healthcare associated infections in the top ten leading causes of death in the United States. Annually, healthcare associated infections account for an estimated 1.7 million infections in hospitals, 99,000 associated deaths, and 4.5 to 5.7 billion dollars in added patient care costs.

The reduction of healthcare associated infections depends upon awareness and adherence to aseptic technique when handling medical equipment that comes into direct contact with a patient. Medical equipment is constantly threatened by exposure to surrounding contaminated surfaces. These surfaces contain microorganisms (bacteria) which can easily adhere to the surface of medical equipment. Once contaminated, the medical equipment becomes a danger to the patient and can serve as a silent killer.

Healthcare institutions use millions of intravenous catheters each year. These catheters are at risk of contamination by a variety of mechanisms. One such mechanism relates to the contamination of the exposed ports of intravenous administration sets. This particular problem arises when an intravenous infusion line is temporarily disconnected from a patient (a process which can occur multiple times per day for an individual patient). During the time that the infusion line is disconnected from the patient, the exposed port of the intravenous line may contact potential contaminants. These contaminants could then lead to infection within a patient's bloodstream.

The critical event in the aforementioned circumstance is the failure to retain the sterility of the intravenous port(s) and failure to adequately disinfect the port in the instance of inadvertent contamination during the time of disconnect from infusion tubing. This risk is, in part, an unanticipated outcome of the somewhat recent implementation of needle-less intravenous systems.

Currently, many practitioners are not actively considering the risk of contamination and are not taking steps to secure the sterility of exposed ports. When efforts are made to maintain the sterility of exposed ports, these efforts are both cumbersome (and therefore at times skipped over), or they fail due to technical shortcomings.

Safe practice recommendations include the use of aseptic technique when handling medical infusion lines. The current aseptic technique, as pertains specifically to intravenous catheters, includes sterilizing the exposed ports used for intermittent infusions with alcohol prep pads between uses. Disinfecting the surfaces of medical equipment with alcohol is a well accepted and established practice. Evidence exists supporting the use of a one minute alcohol exposure as an adequate disinfecting technique.

Current practice often utilizes alcohol cloth swabs to accomplish the task of disinfecting the surface of medical equipment, including intravenous tubing ports. This method has faults limiting its use. The exposure of the port to the cloth swab of alcohol is often performed in variable fashion. With variable techniques and inadequate exposure times to the disinfectant, successful sterilization is unlikely over the entirety of the surface area on female ports. In addition, the current standard disinfecting system of using an alcohol pad exposes the port, to the skin of the practitioner during and immediately after the disinfecting process. Lastly, the alcohol prep pads containing the cloth swabs may not be immediately available for use at the time of greatest need.

The port protection system disclosed in this invention description includes a means to temporarily and safely cover and apply antiseptic to the female end of a medical infusion apparatus. In this way, the device and associated method described will adequately provide a means to maintain the sterility of an indwelling intravenous administration set (or other medical infusion line) that has been disconnected from a patient until it is ready to be reattached for future use.

### SUMMARY OF THE INVENTION

The apparatus of the present invention for sterilizing a tubular medical line port, such as a needleless port, is comprised of a cap having an open tubular cavity on a distal end which is dimensioned to receive the medical line port therein. The cap is provided with a handle on the proximal end for grasping and manipulating the cap.

An outer disinfectant-impregnated member is coaxially secured in the distal cavity and dimensioned for engaging exterior surfaces of a tubular port when inserted into the cavity to be therein retained and disinfected. The handle of the cap is rotated to accordingly scrub the exterior surfaces of the tubular port for sterilization. In one embodiment an inner disinfectant-impregnated member is coaxially secured in the cavity and coaxially spaced and dimensioned relative to the outer member for engaging interior surfaces of the female port when it is inserted into the cavity so that these interior surfaces are also scrubbed with the inner member by rotating the cap.

The distal tubular end of the cap is radially compressible whereby the outer disinfectant-impregnated member may be compressed radially inward for thereby decreasing the spacing between the inner and outer members to apply internal and external scrubbing pressure to a tubular port coaxially inserted between the disinfectant-impregnated members.

A sleeve is coaxially received over the distal end of the cap and is axially slidable thereon from a first position to a second position for thereby axially compressing the tubular distal end of the cap.

The sleeve is axially slid to the second position after the cap has been inserted over the female tubular medical port for temporary securement and sterilization scrubbing within the distal cavity, in order to radially inwardly compress the distal tubular end of the cap. Thereafter, the handle of the cap is then rotated to thereby rotate the disinfectant-impregnated members relative to the interior and exterior surfaces of the female tubular medical port to be sterilized.

When it is time to reconnect the sterilized female tubular medical port for patient use, the sleeve is then axially slid to its first position to thereby loosen the grip of the inner and outer disinfectant-impregnated members and the entire cap is then removed from the sterilized connection for reconnection of the port.

In order to provide radially inward flexibility of the distal end of the cap, the distal tubular end may be provided with annularly spaced and axially or longitudinally extending slots therein for providing radial compression capability.

In a second embodiment of the present invention, the apparatus of the present invention is modified for sterilizing a tubular medical line port which is provided with external threads. In this embodiment, the cavity of the cap is provided with internal threads for threadably mating the port external threads and the outer disinfectant-impregnated member is disposed between these interior threads for thereby scrubbing the external threads of the port when threadably engaged in the cavity of the cap. This outer member is preferably compressible.

In this second embodiment, a disinfectant-impregnated pad is also preferably secured in the cavity of the cap beyond the interior threads whereby a tip of the tubular medical line port to be sterilized will engage the pad when the port is fully threadably secured in the cavity. This pad may be secured to a handle which is rotatably received and retained in the proximal end of the cap whereby the pad may be rotated by the handle for scrubbing the tip when it is fully engaged within the cavity.

### BRIEF DESCRIPTION OF THE DRAWINGS

Other objects and advantages appear hereinafter in the following description and claims, the accompanying drawings show, for the purpose of exemplification, without limiting the scope of the invention or the appended claims, certain practical embodiments of the present invention wherein:

FIG. 1 is a front perspective view illustrating a first embodiment of the apparatus of the present invention for sterilizing a female tubular medical line port as received initially over the top of a female tubular medical line port or IV port;

FIG. 2 is a bottom view of the apparatus shown in FIG. 1 with the IV port removed;

FIG. 3 is a perspective view of the apparatus shown in FIG. 1 with the outer sleeve utilized for compressing the bottom or distal end of the device removed in order to disclose underlying detail;

FIG. 4 is a perspective view of the apparatus shown in FIG. 3 with the inner and outer disinfectant-impregnated scrubbing members removed to expose the interior of the distal end of the cap;

FIG. 5 is an exploded view in front elevation of the apparatus of the present invention shown in vertical mid cross section just prior to being applied over a female IV port;

FIG. 6 is a front view of the structure shown in FIG. 5 with the apparatus of the present invention applied to the female IV port;

FIG. 7 is a front view of the apparatus of the present invention as illustrated in FIG. 6 with the outer sleeve shown in its second position for thereby axially compressing the tubular distal end of the apparatus;

FIG. 8 is a view in front elevation showing a second embodiment of the apparatus of the present invention for sterilizing a tubular medical port having external threads;

FIG. 9 is a perspective view of the apparatus shown in FIG. 8;

FIG. 10 is a view in vertical mid cross section of the apparatus shown in FIG. 8; and

FIG. 11 is a perspective view of a typical threaded medical infusion line port to be sterilized by the apparatus illustrated in FIGS. 8, 9 and 10.

### DETAILED DESCRIPTION OF A PREFERRED EMBODIMENT

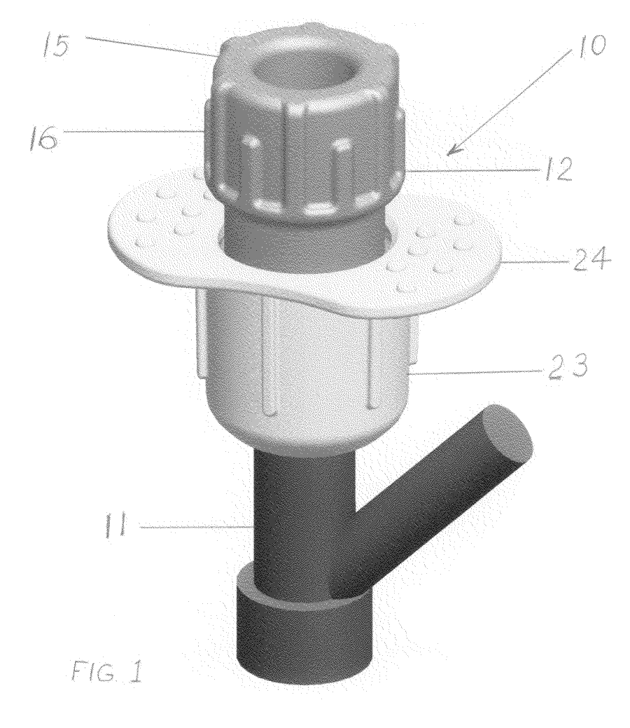

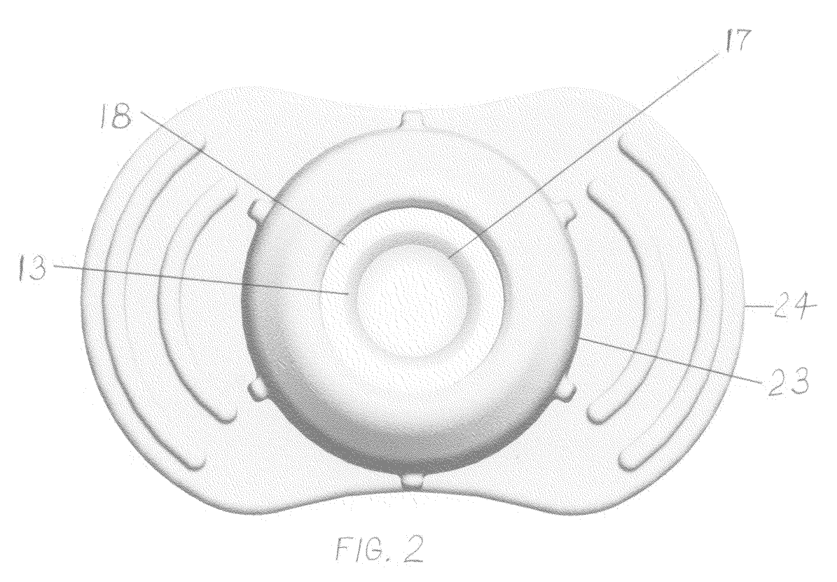

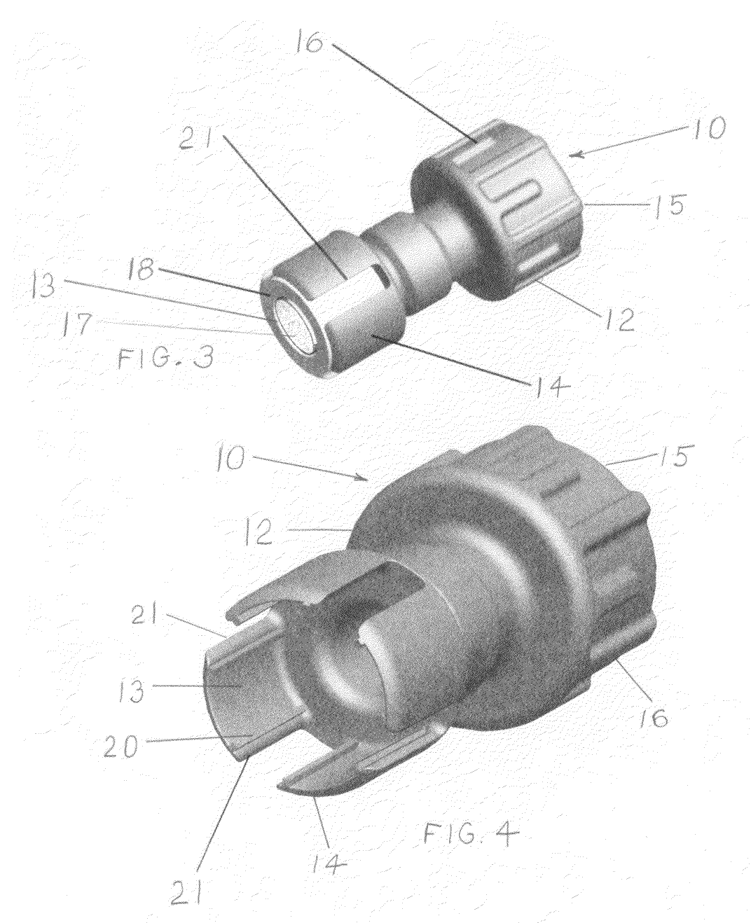

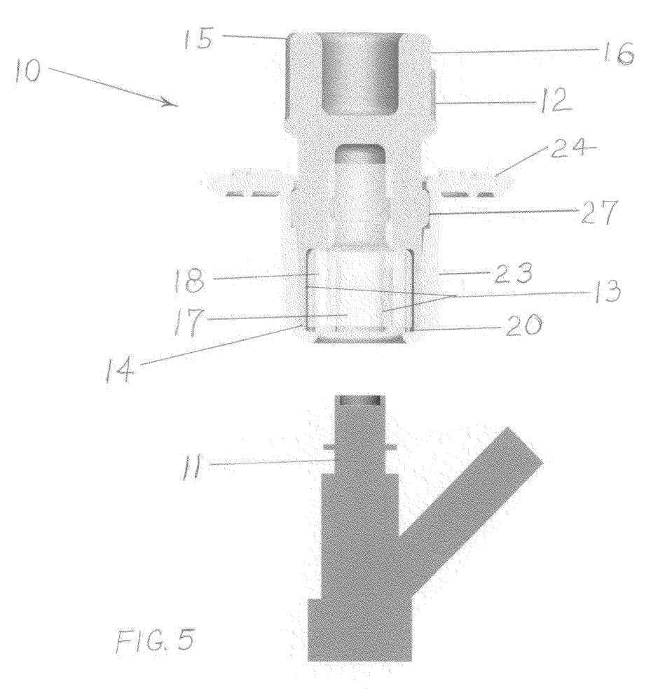

Referring to FIGS. 1 through 8, the apparatus 10 illustrates a first embodiment of the present invention for sterilizing a female tubular medical line port 11 (needleless port) and is comprised of a cap 12 having an open tubular cavity 13 in its tubular distal end 14 for receiving the medical line IV port 11 therein. The proximal end 15 of the cap 12 is provided with a handle 16 for grasping and manipulating the cap 12.

Spaced inner and outer disinfectant-impregnated members 17 and 18 are secured in cavity 13 and dimensioned for respectively engaging interior and exterior surfaces of the female tubular IV port 11 when inserted into the cavity 13. Cavity 13 is further provided with the ribs 20 to assist in engaging outer surfaces of the disinfectant-impregnated member 18 and gripping the same so that when the handle 16 is rotated, outer member 18 will rotate therewith to scrub exterior surfaces of the IV port 11. Inner member 17 in turn is rigidly secured at its upper end to internal parts of cap 12. Inner and outer disinfectant-impregnated members 17 and 18 are here illustrated as being made of a foam which will absorb and retain sterilization fluid, such as gel or liquid alcohol. However, any other suitable absorbent material may be substituted.

Distal end 14 of cap 12 is provided with spaced and longitudinally or axially extending slots 21 in order to render the distal tubular end 14 to be radially compressible whereby the outer disinfectant-impregnated member 18 may be compressed radially inward for thereby decreasing the spacing between the inner and outer members 17 and 18 to apply internal and external scrubbing pressure to the tubular female port 11 inserted between these members.

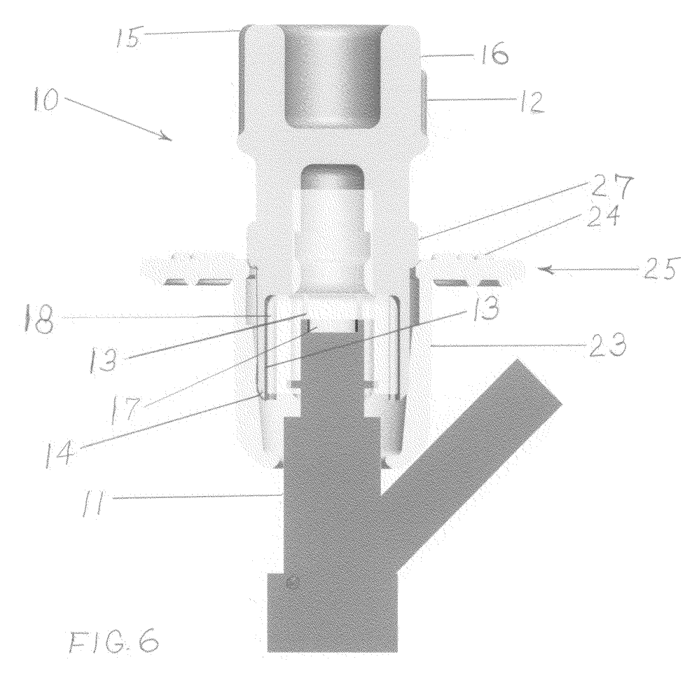

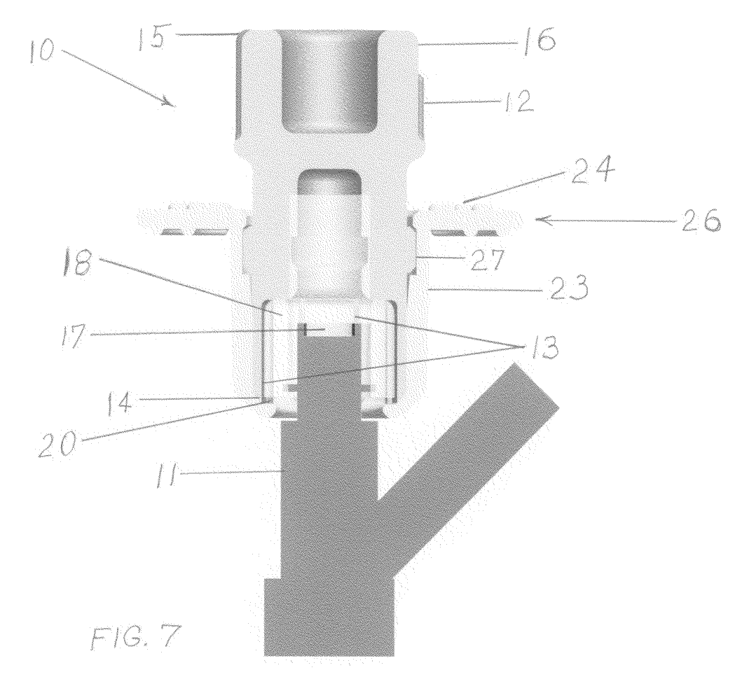

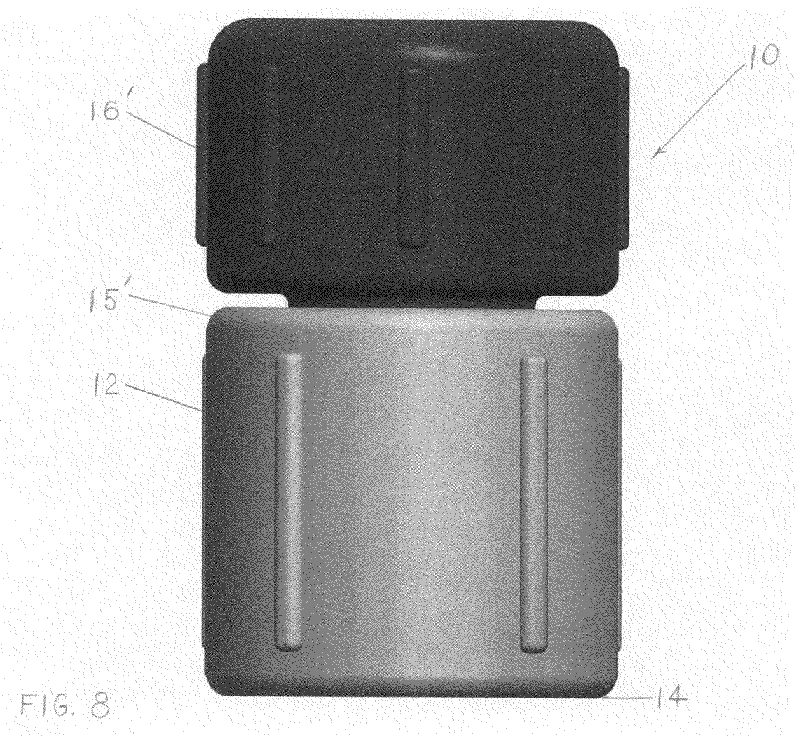

A sleeve 23 is coaxially received over the distal end 14 and is axially slidable thereon from a first position 25 to a second position 26 (see FIGS. 6 and 7) with a snap fit over annular protrusion 27 for thereby radially compressing the tubular distal end 14. To assist in the axial manipulation of sleeve 23, sleeve 23 is provided at its upper end with a protruding handle 24.

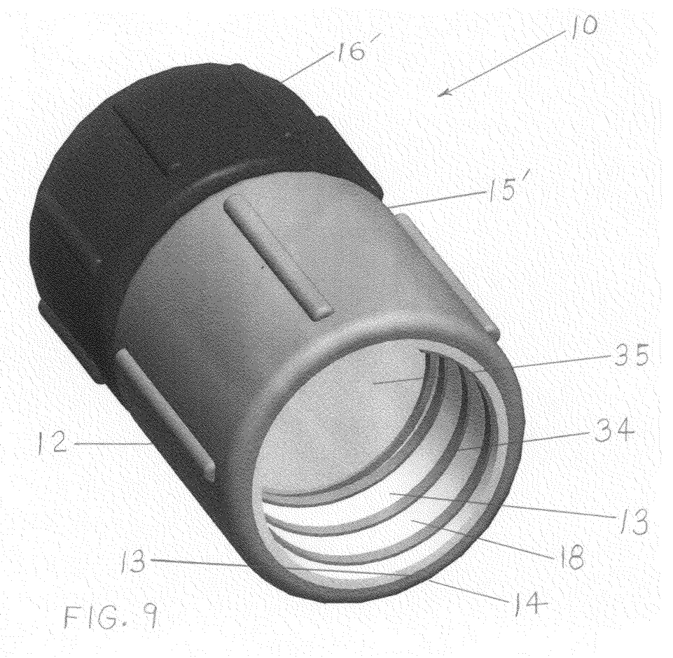

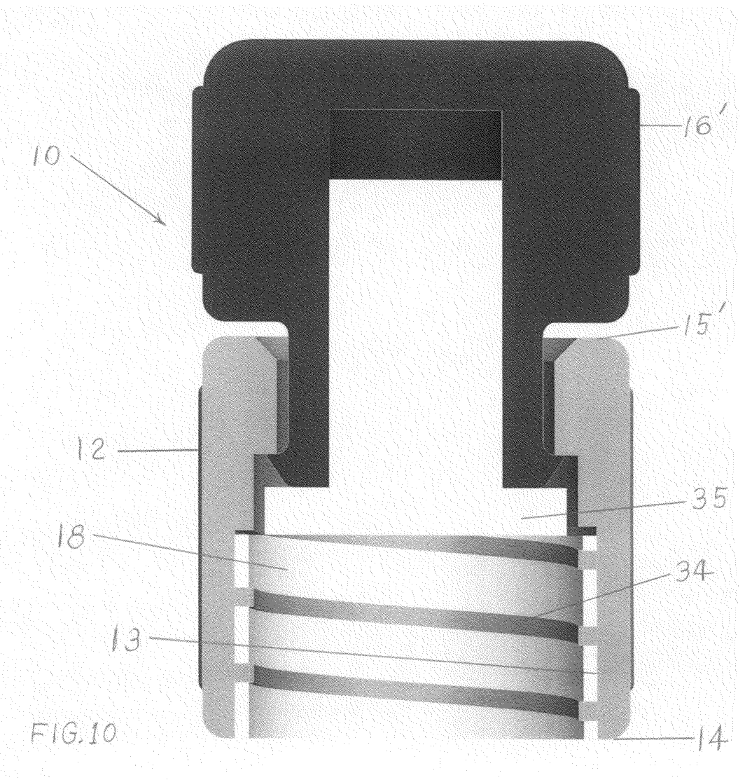

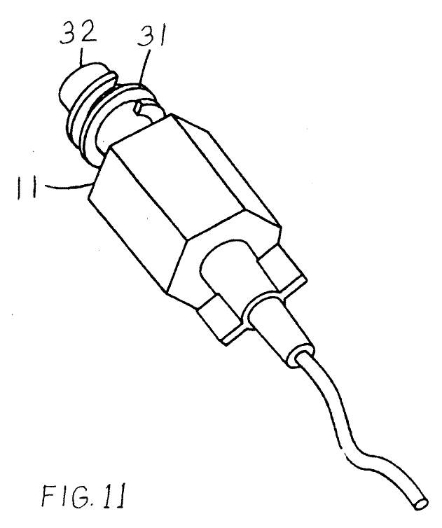

Referring next to the embodiment illustrated in FIGS. 8, 9 and 10, like elements of the first embodiment are designated with the same reference numerals. This embodiment is specifically designed for sterilization of a typical medical line port (see U.S. Pat. No. 6,045,539) which is externally threaded as illustrated in FIG. 11. The port 11 is provided with external threads 31 and the cavity 13 of cap 12 is provided with internal threads 34 for threadably mating the port external threads 31. In this embodiment the outer disinfectant-impregnated member 18 is disposed between interior threads 34 for scrubbing the external threads 31 of port 11 when threadably engaged in cavity 13 of cap 12. The member 18 is preferably compressible to provide a good scrubbing action against thread or threads 31.

A disinfectant-impregnated pad 35 is also secured in cavity 13 beyond interior thread or threads 34 so that the tip 32 of tubular medical line port 11 will engage the pad 35 when the port 11 is fully threadably secured in the cavity 13.

Handle 16′ is rotatably received and retained in the proximal end 15′ of the cap 12, and the pad 35 is secured to the handle 16 interiorly as indicated for rotation with the handle. Accordingly, when the port 11 is fully seated within cavity 13, handle 16′ may be rotated, thereby scrubbing tip 32 of the port 11 with disinfectant-impregnated pad 35.

We claim:

1. An apparatus for sterilizing a tubular medical line port, comprising:
a cap having an open distal tubular end with a cavity therein dimensioned to receive said medical line port therein, an outer disinfectant-impregnated member coaxially secured in said cavity and dimensioned for engaging exterior side surfaces of said tubular port when inserted into said cavity for thereby scrubbing said exterior surfaces with said member by rotating said cap.

2. The apparatus of claim 1, wherein said distal tubular end is radially compressible whereby said outer disinfectant-impregnated member may be compressed radially inward for thereby radially compressing said outer member to apply external scrubbing pressure to a tubular port coaxially inserted between said members.

3. The apparatus of claim 2, including a sleeve coaxially received over said distal end and axially slidable thereon from a first position to a second position for thereby radially compressing said distal tubular end.

4. The apparatus of claim 3, said distal tubular end having annularly spaced and axially extending slots therein for providing said radially compressible capability.

5. The apparatus of claim 1, including an inner disinfectant-impregnated member coaxially secured in said cavity and coaxially spaced and dimensioned relative to said outer member for engaging interior surfaces of said port when inserted into said cavity for also scrubbing said interior surfaces with said inner member by rotating said cap.

6. The apparatus of claim 1, wherein said port is a medical infusion line connector.

7. The apparatus of claim 1, wherein said tubular medical line port to be sterilized is an externally threaded medical line port, said cavity having internal threads for threadably mating said port internal threads, said outer disinfectant-impregnated member being disposed between said interior threads of said cavity for thereby scrubbing said external threads.

8. The apparatus of claim 7, wherein said outer member is compressible.

9. The apparatus of claim 7, including a disinfectant-impregnated pad secured in said cavity beyond said interior threads whereby a tip of said tubular medical line port will engage said pad when said port is fully threadably secured in said cavity.

10. The apparatus of claim 9, including a handle rotatably received and retained in the proximal end of said cap, said pad secured to said handle for rotation therewith for scrubbing said tip.

11. A method of sterilizing a tubular medical line port, comprising:
providing a cap having an open tubular cavity on a distal end dimensioned to receive said medical line port therein, and further having an outer disinfectant-impregnated member coaxially secured in said cavity and dimensioned for engaging exterior side surfaces of said tubular port when inserted into said cavity;
inserting said female tubular port into said cavity and thereby engaging exterior side surfaces of said port with said outer member;
rotating said cap for thereby scrubbing said exterior surfaces with disinfectant; and
removing said distal end from said cavity.

12. The method of claim 11, including providing an inner disinfectant-impregnated member coaxially secured in said cavity and dimensioned for engaging interior surfaces of said tubular port when inserted in said cavity, whereby rotation of said cap also scrubs said interior surfaces.

13. The method of claim 11, wherein said port is provided with external threads;
providing said cavity with internal female threads dimensioned for threadably receiving said medical line port therein, and having said outer disinfectant-impregnated material disposed between said internal threads;
threadably inserting said medical line port into said sleeve and thereby scrubbing the threads of said port with said disinfectant-impregnated material.

14. The method of claim 13;
providing a handle rotatably received and retained in the proximal end of said cap, and having a disinfectant-impregnated pad secured to a distal end of said handle whereby said pad is exposed to a proximal interior of said cap; and
rotating said handle after said medical line port has been threadably seated in said cap for thereby scrubbing a tip portion of said medical line port.
